(19) United States
(12) Patent Application Publication
(10) Pub. No.: US 2015/0031772 A1
(43) Pub. Date: Jan. 29, 2015

(54) ADJUSTABLE BIOACTIVE AGENT DISPERSION WITHIN A POLYMERIC COATING

(71) Applicant: BACTERIN INTERNATIONAL, INC., Belgrade, MT (US)

(72) Inventors: Mark Schallenberger, Bozeman, MT (US); Ben Luchsinger, Bozeman, MT (US); Todd Meyer, Bozeman, MT (US); Helena Lovick, Belgrade, MT (US)

(21) Appl. No.: 14/379,204

(22) PCT Filed: Feb. 19, 2013
(86) PCT No.: PCT/US13/26719

§ 371 Date: Aug. 15, 2014

Related U.S. Application Data
(60) Provisional application No. 61/600,458, filed on Feb. 17, 2012.

(51) Int. Cl. A61L 31/16; A61L 31/10

(52) U.S. Cl. CPC A61L 31/16; A61L 31/10; A61L 2420/02; A61L 2420/06; A61L 2300/404
USPC 514/635; 427/2.1; 427/2.26; 427/2.3; 427/2.24

(57) ABSTRACT

A coating comprised of a polymeric material and a bioactive agent where the properties of the casting solvent dictate the dispersion of the embedding bioactive agent. In preferred embodiments, controlled release of the bioactive agent is achieved by homogeneous dispersion of the bioactive agent within the coating. A further aspect of the invention is directed to formulations where the heterogeneous dispersion of the bioactive agent provides an initial rapid release of the bioactive agent. Methodology for production of coating formulations of the desired properties and the application of the coating to an article are also described.

### CROSS REFERENCE TO RELATED APPLICATIONS

This application claims priority under 35 U.S.C. §119(e) to U.S. Provisional Patent Application Ser. No. 61/600,458 filed Feb. 17, 2012, which is incorporated herein in its entirety by reference.

### FIELD OF THE INVENTION

This invention relates to polymeric coating formulations, methods for making the same and methods for using the same. The polymeric coating includes an embedded bioactive agent, which may be distributed throughout the polymer matrix in an adjustable manner. In one embodiment the coating formulation provides a homogeneous dispersion of the bioactive agent within the resultant coating leading to a minimal initial burst and the sustained release of said agent. Alternatively a heterogeneous dispersion of the bioactive agent within the coating is achievable providing a more rapid initial release of the agent with less sustained release of the agent.

### SUMMARY OF THE INVENTION

For patients with an implanted medical device, implant-associated infections remain a significant risk. In order to minimize the risk of bacterial and fungal-related illness a variety of antimicrobial/bioactive agents have been employed at home and in the clinical setting. Although selected agents have proven abilities to limit disease and inhibit microbial growth, there remains a need for improved infection control.

In some instances, it is preferred that an antimicrobial agent is released quickly since the chance of infection may likely occur soon after implantation of a medical device. Thus, one aspect of the invention is a coating material that delivers an antimicrobial agent to a wound site quickly after implantation, a method for making the coating material, a method for using the coating material on a medical device, and the coated medical device.

In some instances, it is preferred that an antimicrobial agent is released gradually and constantly. Thus, one aspect of the invention is a coating material that delivers an antimicrobial agent to a wound site gradually and constantly, a method for making the coating material, a method for using the coating material on a medical device, and a coated medical device.

In some embodiments, a combination of a heterogeneous coating and a homogeneous coating may be used.

While several methods of coating medical devices with bioactive agents exist, it remains particularly advantageous to obtain coatings with improved properties of uniformity, consistency, and elution kinetics. It is therefore commercially desirable to provide a coating formulation with adjustable physical properties to allow modification of the resultant film.

Another aspect of the present invention is a coating composition used to generate antimicrobial-coated medical devices. In an embodiment, the coating formulation comprises a mixture of a carrier solvent, which eventually evaporates, a polymeric carrier and a bioactive agent that when cast onto a surface of a substrate exhibits homogeneous distribution of the bioactive agent. Another embodiment of the present invention comprises a mixture of a carrier solvent, which eventually evaporates, a polymeric carrier and a bioactive agent that when cast onto a surface exhibits heterogeneous distribution of the bioactive agent.

This invention provides the ability to control the distribution of a bioactive agent throughout a polymer film allowing for adjustable release of said agent from coated medical devices. Disclosed are exemplary methods and formulations for obtaining the adjustable coatings. Such methods and formulations may be applied to provide antimicrobial coatings upon medical devices with selected elution profiles.

### BRIEF DESCRIPTION OF THE DRAWINGS

FIG. 1 provides a detailed explanation of lactide-glycolide polymer nomenclature;

FIG. 2 illustrates a graphical representation of the total amount of chlorhexidine eluted for four different coatings;

FIG. 3 illustrates a graphical representation of elution rates for several samples over a period of seven days;

FIG. 4 illustrates a graphical representation of elution rates for two samples over a period of 70 days; and

FIG. 5 illustrates elution curves for the samples in Table 5 over a period of 5 days.

### DETAILED DESCRIPTION OF THE INVENTION

The present invention is directed to an antimicrobial coating comprising a biodegradable polymeric carrier and a bioactive agent. This coating may be used to coat surfaces of medical devices to inhibit microbial growth and/or colonization. The present invention comprises a desirable composition due to the adjustable nature where the bioactive agent's distribution is dependent on the carrier solvent used in the coating formulation.

As used herein, the term “elution” refers to the release of a bioactive agent during exposure of the coating to a solvent such as water, aqueous buffer, or other simulated biological solution. The term “biodegradable,” as used herein, refers to matter capable of being broken down by biological or environmental processes.

The polymers or copolymers which may be used for this invention specifically relate to biodegradable polymer materials. Examples of the polymer materials or copolymer materials which may be used for this invention include, but are not limited to, polycaprolactones, polyethylene glycols, polyhydroxyalkanoates, polyesteramides, polylactides, polyglycolides, poly(lactide-co-glycolide)s, polyorthoesters, polyoxazolines, and polyurethanes. These polymer materials may be used alone or in any combination with each another. Poly(lactide-co-glycolide), hereafter referred to as PLGA, may preferably contain between about 10% by weight to about 90% by weight of lactide and about 90% by weight to about 10% by weight of glycolide. In some embodiments, the lactide may be D-lactide, L-lactide or D,L-lactide. Thus, in some embodiments, the PLGAs comprises poly(D,L-lactide-co-glycolide). In still other embodiments, the polymer material PLGA comprises poly(L-lactide-co-glycolide).

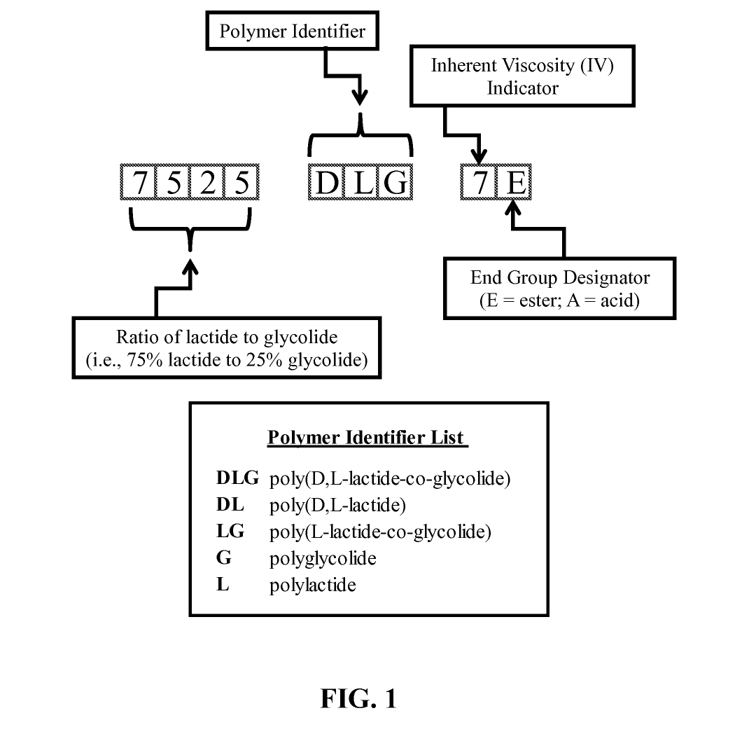

The average molecular weight of the polymer material, for example PLGA, and the ratio of components of the polymer material, for example lactide to glycolide, may be varied to tailor the mechanical, physiochemical, and biodegradable properties of the polymer to the desired ranges. The nomenclature system for the polymer materials is illustrated in FIG. 1. By way of example, FIG. 1 illustrates a polymer material comprising lactide and glycolide. The first numbers, 7525, represent the amount of lactide to glycolide in the polymer—about 75% lactide to about 25% glycolide. The polymer identifier identifies the polymeric material—DLG is poly(D,L-lactide-co-glycolide); D,L is poly(D,L-lactide); LG is poly(L-lactide-co-glycolide); G is polyglycolide and L is polylactide. The Inherent Viscosity (IV) indicator is proportional to the molecular weight of the polymer. The IV values are derived from viscosity measurements of a solution of the polymer at about 0.5% w/v in CHCl₃ at about 30° C. For a polymer of the name 7525 DLG 7E, the second seven indicates an IV of about 0.7 dL/g with a range of about 0.6 dL/g to about 0.8 dL/g. Similarly, a IV indicated by the number 1 would be an IV of about 0.1 dL/g with a range of about 0.0 dL/g to about 0.2 dL/g. An IV indicated by the number 10 would correspond with a IV of about 1.0 dL/g with a range of between about 0.9 dL/g to about 1.1 dL/g. A larger inherent viscosity indicates a higher molecular weight polymer. If the polymer is a PLGA, then preferably the IV range is between of about 0.1 dL/g to about 1.0 dL/g, in some embodiments, the IV range is between about 0.4 dL/g to about 0.8 dL/g.

In the various compositions of the present invention, the polymer material may comprise between about 1% by weight to about 30% by weight, about 5% by weight to about 25% by weight, or about 10% by weight to about 20% by weight to volume of the carrier solvent. In some embodiments, the weight percentage of the polymer material in the dried coating may be about between about 50% to about 99.99%, between about 65% to about 98%, between about 70% to about 95%, or about 75%.

An aspect of the invention comprises a coating formulation. In the various embodiments of the present invention the coating formulation comprises a polymeric material and bioactive agent mixed with a carrier solvent. It is understood that the polymeric material and/or bioactive agent may be added to the carrier solvent in any order. Suitable carrier solvents for the present invention may be any carrier solvent or combination of carrier solvents or mixtures comprising carrier solvents in which the polymeric material and/or the bioactive agent(s) are at least partially soluble. In some embodiments, the polymeric material and/or the bioactive agents are fully soluble in the carrier solvent. Carrier solvents may include, but are not limited to, acetone, acetonitrile, chloroform, diethyl ether, dimethylacetamide, dimethylformamide, dimethylsulfoxide, ethanol, ethyl acetate, hexafluoroisopropanol, hexane, methanol, methylene chloride, tetrahydrofuran, toluene, water and any combinations of two or more of the foregoing.

Various embodiments of the present invention include at least one bioactive agent, which may include a biguanide, an antibiotic, a bioactive component, a biocompatible mineral and combinations thereof. Bioactive agents which may be used with this invention specifically include, but are not limited to, biguanides such as chlorhexidine (as a salt or free base) and polyhexamethylene guanide, salts thereof and combinations thereof. In the present invention, chlorhexidine may be used as its free base, hereafter referred to as CHX.

In various embodiments of the present invention, the bioactive agent may comprise, or additionally comprise, a variety of antibiotics. Antibiotics which may be incorporated into the coating as the bioactive agent including, but not limited to, antifolates, aminoglycosides, carbapenems, cephalosporins, fluoroquinolines, glycopeptides, macrolides, monobactams, oxazolidones, penicillins, rifamins, sulfonamides, tetracyclines and combinations thereof. Antibiotics of particular interest for inclusion within the coatings include, but are not limited to, clindamycin, gentamicin, minocycline, rifampin, tobramycin, vancomycin and combinations thereof.

Bioactive agents may also include, but are not limited to, silver nanoparticles, silver nitrate, silver oxide, silver salts, silver sulfadiazine, silver zeolites, triclosan and combinations thereof. Other bioactive additives such as hormones, growth factors, and/or cells may also be included in the coating. Bioactive agents may be added individually or in combination with other bioactive agents.

Bioactive agents in the coating formulation may include at least one biocompatible mineral including, but not limited to, bioglasses, hydroxyapatites, phosphates, sulfates and combinations thereof.

In the various compositions of the present invention, the bioactive agent may comprise from about 0.01% by weight to about 50% by weight, about 2% by weight to about 35% by weight, or about 5% by weight to about 30% by weight to weight of the polymer. In some embodiments, the bioactive agent is about 25% by weight to weight of the polymeric material in the coating formulation.

In various embodiments of the present invention, the coating formulation comprises the polymer material and the bioactive agent being dissolved in a carrier solvent or mixture wherein the bioactive agent and the solvent do not substantially interact. This formulation provides a homogeneous dispersion of the bioactive agent in the coating while the carrier solvent is present in the coating formulation and after the carrier solvent has evaporated leaving the coating. Though not wanting to be bound by theory, it is believed that the carrier solvents in homogeneous coatings do not substantially interact with the bioactive agent. Thus, the bioactive agent is not pulled to the surface of the coating as the carrier solvent evaporates. Rather, the bioactive agent is homogenously dispersed throughout the coating. Homogeneous coatings exhibit sustained, controlled, gradual and/or constant release of the bioactive agent during elution without a large initial burst of the agent. In some embodiments, the elution rate of homogeneous coatings may be between about 0.01 μg/cm²/day to about 1000 μg/cm²/day with an initial modest burst of about 1% to 10% of the total amount of the bioactive agent. In some embodiments, the bioactive agent is CHX and the polymer material is PLGA, which are dissolved in a carrier solvent or mixture. Examples of carrier solvents that do not substantially interact with the bioactive agent include, but are not limited to, acetonitrile, chloroform, ethyl acetate, methylene chloride, ethylene dichloride, tetrachloromethane, and combinations thereof. In some embodiments, the bioactive agent is CHX and the polymeric material is PLGA and the carrier solvent is acetonitrile.

In some embodiments of the present invention, the coating formulation comprises a polymeric material and a bioactive agent dissolved in a carrier solvent or mixture. The carrier solvent and the bioactive agent substantially interact to produce a heterogeneous coating. The substantial interaction may be through an interaction selected from the group consisting of hydrogen bonding, dipole-dipole interactions, ionic-dipole interactions, ionic-ionic interactions and combinations thereof. Though not wanting to be bound by theory, it is believed that the carrier solvents in heterogeneous coating formulations interacts with elements in the bioactive agent, for example the nitrogens of chlorhexidine may interact with the carrier solvents, through hydrogen bonding. As the carrier solvent evaporates from the coating formation, the bioactive agent is pulled toward the surface of the coating due to these interactions. Thus, the carrier solvents in heterogeneous coatings leave disproportionate amounts of the bioactive agent on or near the surface of the coating. Specific examples of different carrier solvents and bioactive agents, and the bonds between the carrier solvents and the bioactive agents, include but are not limited to, hydrogen bonding accepting solvents and bioactive agents with a hydrogen bonding donor group such as Water:Triclosan; polar solvents with a polar bioactive agent such as Methanol:Gentimicin; polar solvents with an ionic bioactive agent such as Water:Silver; and ionic solvents with an ionic bioactive agent such as Ionic Liquid:Silver. Heterogeneous coatings exhibit localization of the majority of bioactive agent on the coating surface and exhibit a greater initial burst of the bioactive agent and less sustained release of bioactive agent during elution. In some embodiments, the elution rate of heterogeneous coatings may be between about 0.01 μg/cm²/day to about 1000 μg/cm²/day with an initial burst of about 10% to 75% of the total amount of the bioactive agent. Examples of carrier solvents that substantially interact with the bioactive agent include, but are not limited to ethanol, methanol, water and combinations thereof. In some embodiments, the bioactive agent is CHX, the polymeric material is PLGA and the carrier solvent is a mixture of chloroform and methanol in a ratio of 9:1.

In some embodiments, a combination of a heterogeneous coating and a homogeneous coating may be used. In these embodiments, the heterogeneous coating is applied to the homogeneous coatings to provide for both a large initial burst of the bioactive agent followed by a prolonged supply of the bioactive agent. In still other embodiments, multiple layers of the heterogeneous coating and homogeneous coating may be used in any desirable order.

In some embodiments, the coating, either a homogenous coating or a heterogeneous coating, is between about 0.1 microns to about 500 microns thick. In other embodiments, the coating is about 1 micron to about 15 microns. In a further embodiment, the coating is about 2 microns to about 5 microns.

Another aspect of the invention is a process to manufacture a coating. A coating formulation is generated by adding the polymeric carrier and bioactive agent into a carrier solvent or carrier solvent mixture. The resultant mixture is stirred at between about 0° C. to about 75° C., in some embodiments about 40° C. until at least a portion, if not all, of the solids present in the mixture are dissolved. The coating formulation is cooled to between about 0° C. to about 50° C., in some embodiments about 22° C. The article is coated in the coating formulation. After removal of the article from the coating formulation, the carrier solvent is evaporated from the article at temperatures between about 0° C. to about 50° C., in some embodiments under ambient conditions, for a sufficient period to substantially evaporate the carrier solvent, in some embodiments between about 1 second to about 96 hours, in some embodiments between about 24 hours to about 48 hours, depending upon the evaporation conditions. The pressure during evaporation may be ambient or reduced pressure.

Articles to be coated may include metal articles. Metal articles may be pre-treated by various standard methods (e.g., acid etching, sonication, and passivation). Other suitable materials for the articles include, but are not limited to, plastics, elastomers, glasses, tissues, and combinations thereof. Articles may be coated by submersion into the coating formulation followed by withdrawal from the coating formulation at a controlled rate. In some embodiments, the controlled rate is between about 0.1 cm/sec to about 10 cm/sec. In some embodiments, the control rate is about 1.0 cm/sec. Alternatively, the coating formulation may be applied using any suitable method including, but not limited to, dipping, submersion, spraying, painting, and combinations thereof. Articles may be a medical device selected from the group consisting of orthopedic implants, catheters, endotracheal tubes, wound drains, pacemakers, portacaths, stents, any other medical device manufactured from metal, glasses, tissue, elastomers, plastics, and combinations thereof. Specific examples of medical devices include an implantable medical device, an orthopedic device, an implantable orthopedic device, an orthopedic screw, a K-wire, an implantable tissue, and a bone substitute, and combinations thereof.

Another aspect of the invention is a method for coating a medical device. The method comprises casting upon a medical device a coating formulation comprising a biodegradable polymeric material, a bioactive agent, and a solvent. The coating formulation is applied to the medical device and the solvent is evaporated from the coating formulation to form a coating on the medical device. In some embodiments, the coating formation may be homogeneous. In some embodiments, the coating formation may be heterogeneous.

Another aspect of the invention is a method for preparing a homogeneous coating. The method comprises preparing a mixture by adding a polymeric material and a bioactive agent to a carrier solvent, wherein the carrier solvent is selected from the group consisting of acetonitrile, chloroform, ethyl acetate, methylene chloride, ethylene dichloride, tetrachloromethane, and combinations thereof. The mixture is agitated at a temperature between about 0° C. and about 75° C. until a substantial portion, if not all, of the polymeric material and the bioactive agent have dissolved. The mixture is then cooled to form the homogeneous coating.

Any suitable method may be used to agitate the mixture. By way of example, agitation may be accomplished by mechanical stirring, magnetic stirring, shaking, ultrasonication, homogenizing, vortexing and combinations thereof.

In some embodiments, the cooling step may occur at a temperature between about 0° C. and about 50° C. In some embodiments, the cooling step may occur at about 0° C., about 5° C., about 10° C., about 15° C., about 20° C., about 25° C., about 30° C., about 35° C., about 40° C., about 45° C., and about 50° C.

Another aspect of the invention is a coating. The coating comprises a biodegradable polymeric material, and a bioactive agent, wherein the dispersion of the bioactive agent in the coating is homogeneous.

An aspect of the invention is a coated medical device. The coated medical device comprises a medical device and a coating on the medical device. The coating on the medical device comprises a biodegradable polymeric material and a bioactive agent, wherein the bioactive agent is homogeneously dispersed in the coating.

Another aspect of the invention is a method for preparing a heterogeneous coating. The method comprises preparing a mixture by adding a polymeric material and a bioactive agent to a carrier solvent, wherein the carrier solvent or mixture, wherein the carrier solvent or mixture comprises a solvent in whole or in part selected from the group consisting of ethanol, methanol, water and combinations thereof. The mixture is agitated at a temperature between about 0° C. and about 75° C. until a substantial portion, if not all, of the polymeric material and the bioactive agent have dissolved. The mixture is then cooled to form the heterogeneous coating.

Any suitable method may be used to agitate the mixture. By way of example, agitation may be accomplished by mechanical stirring, magnetic stirring, shaking, ultrasonication, homogenizing, vortexing and combinations thereof.

In some embodiments, the cooling step may occur at a temperature between about 0° C. and about 50° C. In some embodiments, the cooling step may occur at about 0° C., about 5° C., about 10° C., about 15° C., about 20° C., about 25° C., about 30° C., about 35° C., about 40° C., about 45° C., and about 50° C.

Another aspect of the invention is a coating. The coating comprises a biodegradable polymeric material, and a bioactive agent, wherein the dispersion of the bioactive agent in the coating is heterogeneous.

An aspect of the invention is a coated medical device. The coated medical device comprises a medical device and a coating on the medical device. The coating on the medical device comprises a biodegradable polymeric material and a bioactive agent, wherein the bioactive agent is heterogeneously dispersed in the coating.

### Example 1

In order to produce a polymeric coating with a homogeneous dispersion of CHX, 7525 DLG 7E PLGA (750 mg, 15.0% w/v) was combined with CHX (75 mg, 10.0% w/w) in CH₃CN (5.0 mL) (Coating A, Table 1). The mixture was stirred thoroughly at about 40° C. until all solids dissolved. The resultant solution was allowed to cool to 22° C. Stainless steel rods were dipped into the solution (37 mm depth) and removed at a controlled withdrawal rate after which the carrier solvent was allowed to evaporate from the rods under ambient conditions for between about 24 to about 48 hours. Coatings obtained using this formulation and method were a calculated to be about 1.20 μm thick with an average measured CHX total load of 16.3 μg/cm².

### Example 2

An alternative polymeric coating with a homogeneous dispersion of CHX utilized 5050 DLG 4.5A PLGA (744 mg, 14.9% w/v) combined with CHX (74 mg, 10.0% w/w) in CH₃CN (5.0 mL) (Coating B, Table 1). The formulations and coated articles were prepared as described in Example 1. Coatings obtained by this method were calculated to be about 1.20 μm thick with an average measured CHX total load of 15.6 μg/cm².

### Example 3

In order to produce a polymeric coating with a heterogeneous dispersion of CHX, 7525 DLG 7E PLGA (899 mg, 17.6% w/v) was combined with CHX (90 mg, 10.0% w/w) in CHCl₃/MeOH (9:1, 5.0 mL) (Coating C, Table 1). The mixture was stirred thoroughly at 40° C. until all solids were dissolved. The resultant solution was allowed to cool to 22° C. Stainless steel rods were dipped into the solution (39 mm depth) and removed at a controlled withdrawal rate after which the carrier solvent was allowed to evaporate from the rods under ambient conditions for between about 24 to about 48 hours. Coatings obtained using this formulation and method were calculated to be about 1.20 μm thick with an average measured CHX total load of 15.7 μg/cm².

### Example 4

An alternative polymeric coating with a heterogeneous dispersion of CHX utilized 5050 DLG 4.5A PGLA (899 mg, 18.0% w/v) combined with CHX (90 mg, 10.0% w/w) in CHCl₃/MeOH (9:1, 5.0 mL) (Coating D, Table 1). The formulations and coated articles were prepared as described in Example 3. Coatings obtained by this method were about 1.41 μm thick with an average measured total load of 18.3 μg/cm² of CHX. Table 1 summarizes characteristics of Coatings A-D.

TABLE 1
Formulation Compositions

| | Type of | Amt. of | Amt. of | |
|---|---|---|---|---|
| Coating | PLGA | PLGA | CHX | Carrier Solvent |
| A | 7515 DLG 7E | 750 mg | 75 mg | CH₃CN (5.0 mL) |
| B | 5050 DLG 4.5A | 744 mg | 74 mg | CH₃CN (5.0 mL) |
| C | 7515 DLG 7E | 880 mg | 88 mg | CHCl₃:MeOH; |
| | | | | 9:1 (5.0 mL) |
| D | 5050 DLG 4.5A | 899 mg | 90 mg | CHCl₃:MeOH; |
| | | | | 9:1 (5.0 mL) |

### Study of the Elution Profiles of the Antimicrobial Coatings According to the Invention

Four separate coating formulations were examined for their elution characteristics in water. Stainless steel rods were coated with Coatings A-D (Table 1) as described in Examples 1-4. The initial amount of CHX on the rods was measured by UV absorbance of dissolved films. The initial amount of CHX on the rods for all the coatings was between about 87 μg to about 107 μg or 15.6-18.3 μg/cm² (Table 2). The rods were subsequently soaked in water at 37° C. for 24 hours. After a single day of elution, the amount of CHX released from the metal rods was considerably higher for the coatings cast using CHCl₃:MeOH (Coatings C and D) than for coatings cast using CH₃CN (Coatings A and B) (Table 2). This discrepancy illustrates the varied distribution of the CHX within the antimicrobial coating for different coating types.

TABLE 2

| | Initial Total Load of CHX versus Day 1 Elution of the Four Formulations | | | | |
|---|---|---|---|---|---|
| | | | Initial load | CHX mass per | Amount of CHX |
| Coating | Carrier Solvent | Polymer | CHX (μg) | area (μg/cm²) | eluted in 1 day (μg) |
| A | CH₃CN | 7525 DLG 7E | 91.12 | 16.3 | 3.77 |
| B | CH₃CN | 5050 DLG 4.5A | 87.12 | 15.6 | 6.36 |
| C | CHCl₃:MeOH (9:1) | 7525 DLG 7E | 94.42 | 15.7 | 37.08 |
| D | CHCl₃:MeOH (9:1) | 5050 DLG 4.5A | 107.42 | 18.3 | 45.69 |

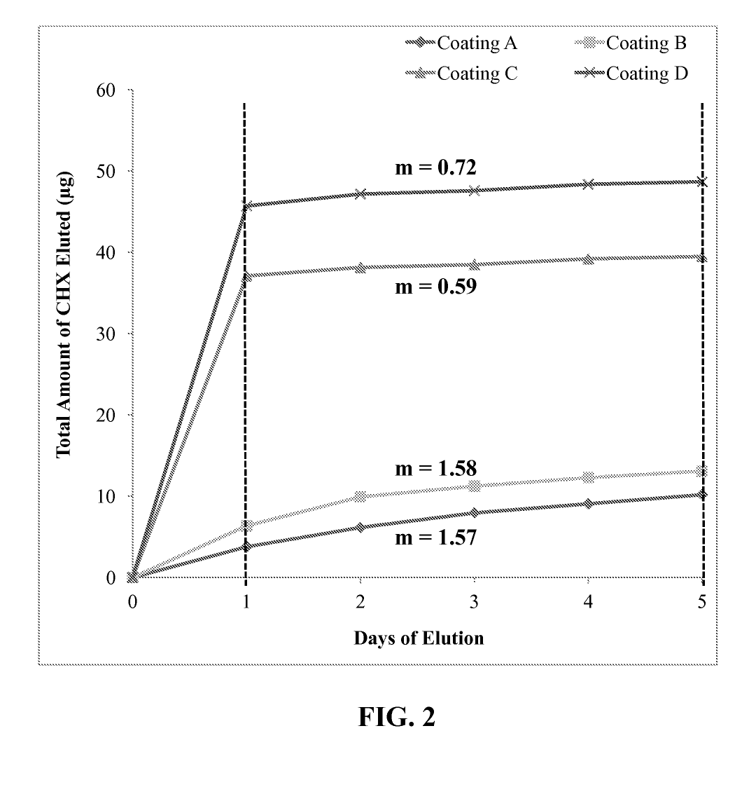

In addition, the coatings that exhibited a sustained controlled release of the antimicrobial agent (Coatings A and B) provided a higher rate of release of CHX from day 1 to day 5 (Table 3, elution slopes of 1.57 and 1.58, respectively). The coatings that exhibited a rapid release of CHX on day 1 (Coatings C and D) provided a slower subsequent release of CHX (Table 3, elution slopes of 0.59 and 0.72, respectively). For each formulation, the total amount of CHX eluted over time is illustrated in FIG. 2. The elution slope for each coating was calculated by the linear regression of the cumulative amount of CHX eluted from day 1 to day 5.
Coating formulations cast with CH₃CN provided more homogeneous dispersion of CHX, as determined by the elution profile (Coatings A and B, Table 3). The elution behavior of the coatings was consistent between Coatings A and B even though different PLGA formulations were used. Similarly, coatings cast in a mixture of CHCl₃ and MeOH provided a heterogeneous dispersion of CHX regardless of PLGA type (Coatings C and D). These results illustrate that the type of carrier solvent used in the coating formulation dictates the distribution of the CHX within the coating and not the PLGA formulation.

TABLE 3
Daily Amount of CHX Eluted (μg)

| | Day: | | | | | slope, m |
|---|---|---|---|---|---|---|
| Formulation | 1 | 2 | 3 | 4 | 5 | days (1-5) |
| Coating A | 3.77 | 2.38 | 1.79 | 1.12 | 1.12 | 1.57 |
| Coating B | 6.36 | 3.56 | 1.32 | 1.04 | 0.81 | 1.58 |
| Coating C | 37.08 | 1.05 | 0.35 | 0.71 | 0.29 | 0.59 |
| Coating D | 45.69 | 1.46 | 0.42 | 0.79 | 0.31 | 0.72 |

### Example 5

Elution curves were generated for various samples where there were varied amounts of chlorhexidine. The polymer in each of the samples was 7525 DLG 7E PLGA.

The samples were cast in acetonitrile and the elution was measured in a phosphate buffered saline as in Example 1. The concentration of polymer of each was set to generate approximately a 2.0 μm thick coating. Table 4 illustrates the amount 7525 DLG 7E PLGA, CHX, and acetonitrile used in the dipping solution to generate each coating. Table 4 also illustrates the initial total amount of CHX present on the articles and the amount eluted after 1 day in terms of μg CHX and percent of total.

TABLE 4

| Sample | | | | | Amount of |
|---|---|---|---|---|---|
| (% w | Amount of | Amount of | Amount of | | CHX |
| CHA/w of | PLGA in | CHX in | CH₃CN in | Total | Eluted |
| PLGA) | Dipping | Dipping | Dipping | Amount of | in 1 Day |
| Cast in | Solution | Solution | Solution | CHX on | μg (% of |
| CH₃CN | (grams) | (grams) | (mL) | Articles | total) |
| 5% CHX | 1.06 | .05 | 7.0 | 68.0 | 2.44 (3.6%) |
| 10% CHX | 1.23 | .12 | 7.0 | 126.1 | 3.34 (2.7%) |
| 15% CHX | 1.39 | .21 | 7.0 | 182.3 | 6.84 (3.8%) |
| 20% CHX | 1.80 | .36 | 7.0 | 286.0 | 12.37 (4.3%) |
| 25% CHX | 1.76 | .44 | 7.0 | 322.2 | 20.31 (6.3%) |
| 30% CHX | 1.79 | .54 | 7.0 | 383.5 | 28.10 (7.3%) |
| 35% CHX | 1.79 | .63 | 7.0 | 456.3 | 35.81 (7.9%) |

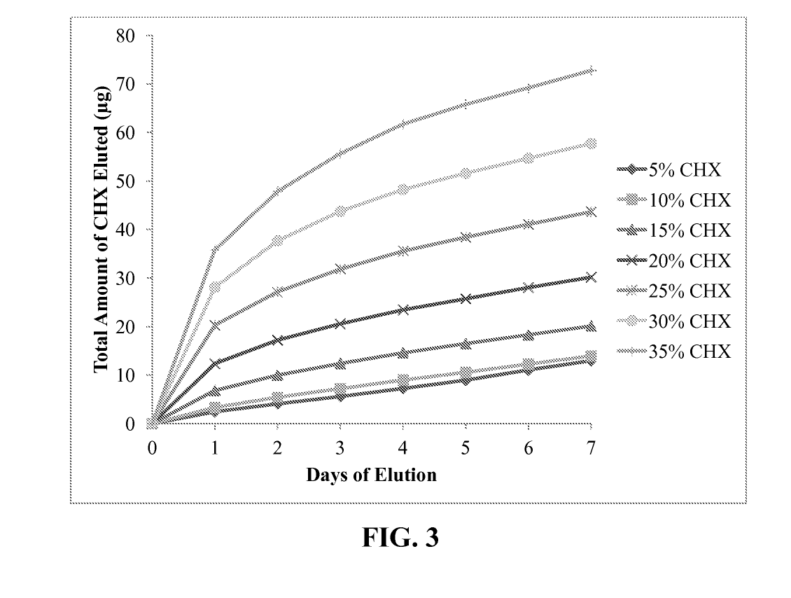

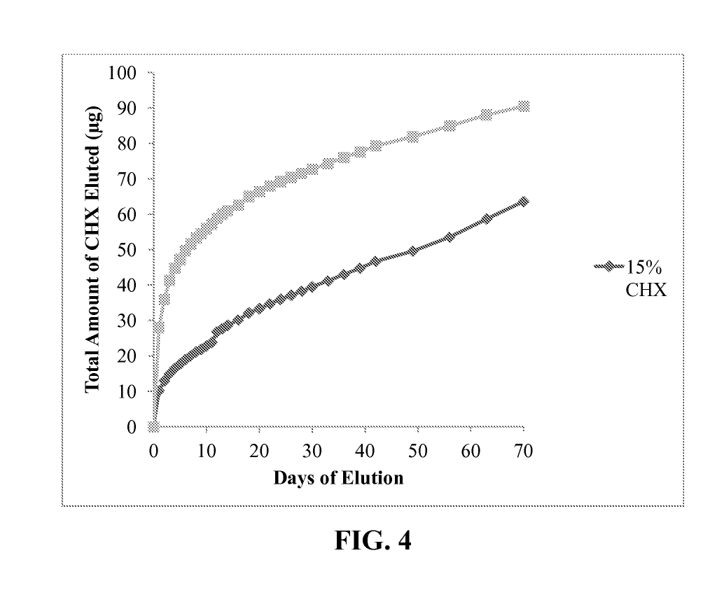

FIG. 3 illustrates elution curves for the samples in Table 4 over a period of 7 days. FIG. 3 illustrates that the elution of chlorhexidine were sustained for various coating with various concentrations of chlorhexidine. FIG. 4 illustrates elution curves for two samples, 15% CHX cast in acetonitrile and 25% CHX cast in acetonitrile for a period of about 70 days. FIG. 4 illustrates that the elution of chlorhexidine may be sustained for a long time with a minimal initial elution burst. Rather, the chlorhexidine is homogeneously distributed throughout the coating and therefore elutes the chlorhexidine over a longer period of time.

### Example 6

Elution curves were generated for various samples cast in two additional non-hydrogen bonding solvents, ethyl acetate (EtOAc), and methylene chloride (CH₂Cl₂) and two additional hydrogen bonding solvents, Acetone:H₂O 9:1 and tetrahydrofuran:H₂O 9:1 (THF:H₂O). The polymer in each of the samples was 7525 DLG 7E PLGA and each sample contained 10% w/w chlorhexidine. The samples were cast and the elution was measured in a phosphate buffered saline as in Example 1. The concentration of polymer of each was set to generate approximately a 2.0 μm thick coating. Table 5 illustrates the amount 7525 DLG 7E PLGA, CHX, and solvent used in the dipping solution to generate each coating. Table 4 also illustrates the initial total amount of CHX present on the articles and the amount eluted after 1 day in terms of μg CHX and percent of total

TABLE 5

| | | | | | Amount of |
|---|---|---|---|---|---|
| | Amount of | Amount of | | | CHX |
| | PLGA in | CHX in | Amount of | Total | Eluted in 1 |
| | Dipping | Dipping | Solvent in | Amount of | Day |
| | Solution | Solution | Dipping | CHX on | μg (% of |
| Sample | (grams) | (grams) | Solution (mL) | Articles | total) |
| EtOAC | .71 | .07 | 5.0 | 143.4 | 9.05 (6.3%) |
| CH₂Cl₂ | .74 | .07 | 5.0 | 121.5 | 9.12 (7.5%) |
| Acetone:H₂O | .73 | .07 | 5.0 | 166.9 | 44.80 (26.9%) |
| 9:1 | | | | | |
| THF:H₂O 9:1 | .56 | .06 | 5.0 | 117.6 | 30.3 (25.8%) |

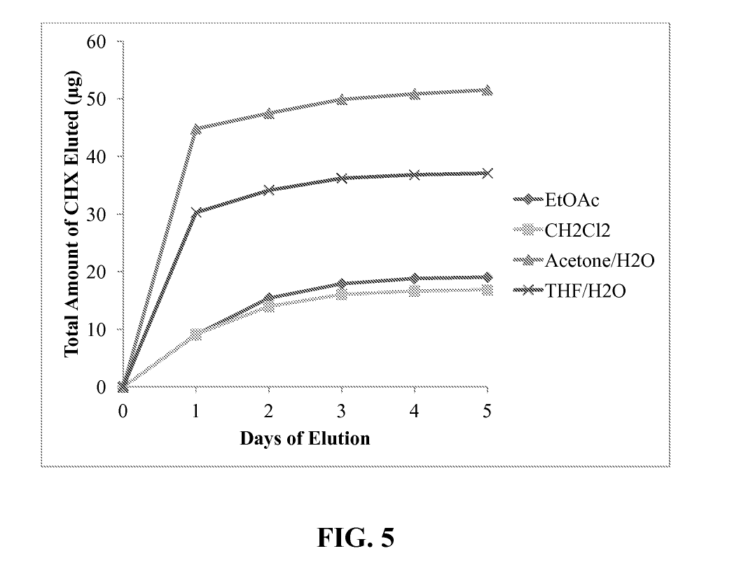

FIG. 5 illustrates elution curves for the samples in Table 5 over a period of 5 days. FIG. 5 illustrates that the elution of chlorhexidine was sustained for coating prepared with non-hydrogen bonding solvents with a minimal initial burst while the coating cast in the hydrogen bonding solvents had a large initial burst and less sustained elution.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A coating formulation for preparing a coating having a homogenous dispersion of a bioactive agent, comprising:
a) a biodegradable polymeric material;
b) the bioactive agent; and
c) a carrier solvent, wherein the bioactive agent and the carrier solvent do not substantially interact.

2. The coating formulation of claim 1, wherein the carrier solvent is selected from the group consisting of acetonitrile, chloroform, ethyl acetate, methylene chloride, ethylene dichloride, tetrachloromethane, and combinations thereof.

3. The coating formulation of claim 1, wherein the biodegradable polymeric material is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

4. The coating formulation of claim 1, wherein the biodegradable polymeric material is poly(lactide-co-glycolide).

5. The coating formulation of claim 4, wherein an inherent viscosity of the poly(lactide-co-glycolide) is between about 0.1 dL/g to about 1.0 dL/g.

6. The coating formulation of claim 4, wherein an inherent viscosity of the poly(lactide-co-glycolide) is between about 0.4 dL/g to about 0.8 dL/g.

7. The coating formulation of claim 4, wherein the poly(lactide-co-glycolide) comprises between about 10% to about 90% of lactide and about 10% to about 90% of glycolide.

8. The coating formulation of claim 7, wherein the lactide is selected from the group consisting of D-lactide, L-lactide, D,L-lactide and combinations thereof.

9. The coating formulation of claim 4, wherein the poly(lactide-co-glycolide) comprises poly(L-lactide-co-glycolide).

10. The coating formulation of claim 1, wherein an amount of the biodegradable polymeric material in the coating formulation is between about 1% and about 30% by weight to volume of the carrier solvent.

11. The coating formulation of claim 1, wherein an amount of the biodegradable polymeric material in the coating formulation is between about 5% and about 25% by weight to volume of the carrier solvent.

12. The coating formulation of claim 1, wherein an amount of the biodegradable polymeric material in the coating formulation is between about 10% and about 20% by weight to volume of the carrier solvent.

13. The coating formulation of claim 1, wherein the bioactive agent is a biguanide.

14. The coating formulation of claim 13, wherein the biguanide is selected from the group consisting of chlorhexidine, polyhexamethylene guanide, salts thereof and combinations thereof.

15. The coating formulation of claim 14, wherein the bioactive agent is a combination of a biguanide and an antibiotic.

16. The coating formulation of claim 1, wherein the bioactive agent is an antibiotic.

17. The coating formulation of claim 1, wherein the bioactive agent is a biocompatible mineral.

18. The coating formulation of claim 1, wherein the bioactive agent is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

19. The coating formulation of claim 1, wherein the bioactive agent is chlorhexidine.

20. The coating formulation of claim 1, wherein the bioactive agent is chlorhexidine free base.

21. The coating formulation of claim 1, wherein the bioactive agent is present in the coating formulation in amounts between about 0.01% to about 50% by weight of the biodegradable polymeric material.

22. The coating formulation of claim 1, wherein the bioactive agent is present in the coating formulation in amounts between about 2% to about 35% by weight of the biodegradable polymeric material.

23. The coating formulation of claim 1, wherein the bioactive agent is present in the coating formulation in amounts between about 5% to about 30% by weight of the biodegradable polymeric material.

24. The coating formulation of claim 1, wherein the bioactive agent is present in the coating formulation in amount of about 25% by weight of the biodegradable polymeric material.

25. The coating formulation of claim 1, wherein the biodegradable polymeric material and the bioactive agent are at least partially soluble in the carrier solvent.

26. The coating formulation of claim 1, wherein the bioactive agent is gradually and constantly released during elution.

27. A method for coating a medical device, comprising:
a. casting upon the medical device a coating formulation comprising:
i. a biodegradable polymeric material;
ii. a bioactive agent; and
iii. a carrier solvent, wherein the bioactive agent and the carrier solvent do not substantially interact;
b. applying the coating formulation to the medical device; and
c. evaporating the carrier solvent from the coating formulation to form a coating on the medical device, wherein dispersion of the bioactive agent in the coating is homogeneous.

28. The method of claim 27, wherein the step of applying the coating formulation is selected from the group consisting of dipping, submersion, spraying, painting, and combinations thereof.

29. The method of claim 27, wherein the step of applying the coating formulation to the medical device is submersion.

30. The method of claim 29, wherein the medical device is submersed in the coating formulation and removed at a controlled rate, wherein the controlled rate is between 0.1 cm/sec to 10 cm/sec.

31. The method of claim 29, wherein the medical device is submersed in the coating formation and removed at a controlled rate, wherein the controlled rate is about 1.0 cm/sec.

32. The method of claim 27, wherein the evaporating step occurs for between about 1 second to about 96 hours and wherein the temperature of the evaporating step is between about 0° C. to about 50° C.

33. The method of claim 27, wherein the evaporation step occurs at ambient pressure.

34. The method of claim 27, wherein the evaporation step occurs at reduced pressure.

35. The method of claim 27, wherein the medical device is selected from the group consisting of orthopedic implants, catheters, endotracheal tubes, wound drains, pacemakers, portacaths, and stents, or any other medical device manufactured from metal, glasses, tissue, elastomers, plastics and combinations thereof.

36. The method of claim 27, wherein a thickness of the coating on the medical device is between about 0.1 microns to about 500 microns.

37. The method of claim 27, wherein the medical device is an implantable medical device.

38. The method of claim 27, wherein the medical device is an orthopedic device.

39. The method of claim 27, wherein the medical device is an implantable orthopedic device.

40. The method of claim 27, wherein the medical device is an orthopedic screw.

41. The method of claim 27, wherein the medical device is a K-wire.

42. The method of claim 27, wherein the medical device is an implantable tissue.

43. The method of claim 27, wherein the medical device is a bone substitute.

44. The method of claim 27, wherein the medical device comprises a material selected from the group consisting of metal, glasses, tissue, elastomers, plastics and combinations thereof.

45. The method of claim 27, wherein the coating eludes the bioactive agent at an elution rate of between about 0.01 μg/cm²/day to about 1000 μg/cm²/day when the medical device is implanted in a patient.

46. The method of claim 27, wherein the biodegradable polymeric material of the coating formulation is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

47. The method of claim 27, wherein the carrier solvent of the coating formulation is selected from the group consisting of acetonitrile, chloroform, ethyl acetate, methylene chloride and combinations thereof.

48. The method of claim 27, wherein the bioactive agent of the coating formulation is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

49. A method for preparing a homogeneous coating, the method comprising:
preparing a mixture by adding a polymeric material and a bioactive agent to a solvent, wherein the carrier solvent is selected from the group consisting of acetonitrile, chloroform, ethyl acetate, methylene chloride, ethylene dichloride, tetrachloromethane and combinations thereof;
agitating the mixture between 0° C. and about 75° C. until at least a portion of the biodegradable polymeric material and the bioactive agent have dissolved; and
cooling the agitated mixture to form the homogeneous coating.

50. The method of claim 49, wherein the agitation step is performed by mechanical stirring, magnetic stirring ultrasonication, shaking, homogenizing, vortexing, or combinations thereof.

51. The method of claim 49, wherein the cooling step occurs at a temperature between about 0° C. and about 50° C.

52. The method of claim 49, wherein the biodegradable polymeric material is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

53. The method of claim 49, wherein the bioactive agent is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

54. A coating, comprising:
a) a biodegradable polymeric material; and
b) a bioactive agent, wherein dispersion of the bioactive agent in the coating is homogeneous.

55. The coating of claim 54, wherein the biodegradable polymeric material is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

56. The coating of claim 54, wherein the biodegradable polymeric material is poly(lactide-co-glycolide).

57. The coating of claim 56, wherein an inherent viscosity of the poly(lactide-co-glycolide) is between about 0.1 dL/g to about 1.0 dL/g.

58. The coating of claim 56, wherein an inherent viscosity of the poly(lactide-co-glycolide) is between about 0.4 dL/g to about 0.8 dL/g.

59. The coating of claim 56, wherein the poly(lactide-co-glycolide) comprises between about 10% to about 90% of lactide and about 10% to about 90% of glycolide.

60. The coating of claim 59, wherein the lactide is selected from the group consisting of D-lactide, L-lactide, D,L-lactide and combinations thereof.

61. The coating of claim 56, wherein the poly(lactide-co-glycolide) comprises poly(L-lactide-co-glycolide).

62. The coating of claim 54, wherein an amount of the biodegradable polymeric material in the coating is between about 1% and about 30% by weight to volume of the carrier solvent.

63. The coating of claim 54, wherein the bioactive agent is a biguanide.

64. The coating of claim 63, wherein the biguanide is selected from the group consisting of chlorhexidine, polyhexamethylene guanide, salts thereof, and combinations thereof.

65. The coating of claim 54, wherein the bioactive agent is selected from the group consisting of a biguanide, an antibiotic, a biocompatible mineral and combinations thereof.

66. The coating of claim 54, wherein the bioactive agent is an antibiotic.

67. The coating of claim 54, wherein the bioactive agent is a biocompatible mineral.

68. The coating of claim 54, wherein the bioactive agent is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

69. The coating of claim 54, wherein the bioactive agent is chlorhexidine.

70. The coating of claim 54, wherein the bioactive agent is chlorhexidine free base.

71. The coating of claim 54, wherein the bioactive agent is present in the coating in amounts between about 0.01% to about 50% by weight of the biodegradable polymeric material.

72. The coating of claim 54, wherein the bioactive agent is present in the coating in amounts between about 2% to about 35% by weight of the biodegradable polymeric material.

73. The coating of claim 54, wherein the bioactive agent is present in the coating in amounts between about 5% to about 30% by weight of the biodegradable polymeric material.

74. The coating of claim 54, wherein the bioactive agent is present in the coating in amount of about 25% by weight of the biodegradable polymeric material.

75. The coating of claim 54, wherein the bioactive agent is gradually and constantly released during elution.

76. A coated medical device, comprising:
a) a medical device; and
b) a coating on the medical device, wherein the coating comprises:
a. a biodegradable polymeric material; and
b. a bioactive agent, wherein dispersion of the bioactive agent in the coating is homogeneous.

77. The medical device of claim 76, wherein the biodegradable polymeric material of the coating is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

78. The medical device of claim 76, wherein the biodegradable polymeric material of the coating is poly(lactide-co-glycolide).

79. The medical device of claim 76, wherein the bioactive agent of the coating is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

80. The medical device of claim 76, wherein the bioactive agent of the coating is gradually and constantly released during elution.

81. The medical device of claim 76, wherein the coating eludes the bioactive agent at an elution rate of between about 0.01 μg/cm²/day to about 1000 μg/cm²/day when the medical device is implanted in a patient.

82. A coating formulation for preparing a coating having a heterogeneous dispersion of a bioactive agent, comprising:
d) a biodegradable polymeric material;
e) the bioactive agent; and
f) a carrier solvent, wherein the bioactive agent and the carrier solvent substantially interact.

83. The coating formulation of claim 82, wherein the bioactive agent and the carrier solvent substantially interact through an interaction selected from the group consisting of hydrogen bonding, dipole-dipole interactions, ionic-dipole interactions, ionic-ionic interactions and combinations thereof.

84. The coating formulation of claim 82, wherein the hydrogen bonding solvent is selected from the group consisting of ethanol, methanol, water and combinations thereof.

85. The coating formulation of claim 82, wherein the biodegradable polymeric material is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

86. The coating formulation of claim 82, wherein the biodegradable polymeric material is poly(lactide-co-glycolide).

87. The coating formulation of claim 86, wherein an inherent viscosity of the poly(lactide-co-glycolide) is between about 0.1 dL/g to about 1.0 dL/g.

88. The coating formulation of claim 86, wherein an inherent viscosity of the poly(lactide-co-glycolide) is between about 0.4 dL/g to about 0.8 dL/g.

89. The coating formulation of claim 86, wherein the poly(lactide-co-glycolide) comprises between about 10% to about 90% of lactide and about 10% to about 90% of glycolide.

90. The coating formulation of claim 89, wherein the lactide is selected from the group consisting of D-lactide, L-lactide, D,L-lactide and combinations thereof.

91. The coating formulation of claim 86, wherein the poly(lactide-co-glycolide) comprises poly(L-lactide-co-glycolide).

92. The coating formulation of claim 82, wherein an amount of the biodegradable polymeric material in the coating formulation is between about 1% and about 30% by weight to volume of the carrier solvent.

93. The coating formulation of claim 82, wherein an amount of the biodegradable polymeric material in the coating formulation is between about 1% and about 25% by weight to volume of the carrier solvent.

94. The coating formulation of claim 82, wherein an amount of the biodegradable polymeric material in the coating formulation is between about 10% and about 20% by weight to volume of the carrier solvent.

95. The coating formulation of claim 82, wherein the bioactive agent is a biguanide.

96. The coating formulation of claim 95, wherein the biguanide is selected from the group consisting of chlorhexidine, polyhexamethylene guanide, salts thereof, and combinations thereof.

97. The coating formulation of claim 82, wherein the bioactive agent is a combination of a biguanide and an antibiotic.

98. The coating formulation of claim 82, wherein the bioactive agent is an antibiotic.

99. The coating formulation of claim 82, wherein the bioactive agent is a biocompatible mineral.

100. The coating formulation of claim 82, wherein the bioactive agent is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

101. The coating formulation of claim 82, wherein the bioactive agent is chlorhexidine.

102. The coating formulation of claim 82, wherein the bioactive agent is chlorhexidine free base.

103. The coating formulation of claim 82, wherein the bioactive agent is present in the coating formulation in amounts between about 0.01% to about 50% by weight of the biodegradable polymeric material.

104. The coating formulation of claim 82, wherein the bioactive agent is present in the coating formulation in amounts between about 2% to about 35% by weight of the biodegradable polymeric material.

105. The coating formulation of claim 82, wherein the bioactive agent is present in the coating formulation in amounts between about 5% to about 30% by weight of the biodegradable polymeric material.

106. The coating formulation of claim 82, wherein the bioactive agent is present in the coating formulation in amount of about 25% by weight of the biodegradable polymeric material.

107. The coating formulation of claim 82, wherein the biodegradable polymeric material and the bioactive agent are at least partially soluble in the carrier solvent.

108. The coating formulation of claim 82, wherein the bioactive agent is substantially rapidly released immediately upon implantation.

109. The coating formulation of claim 82, wherein the carrier solvent is a solvent mixture in a ratio of 9:1 of chloroform to methanol.

110. A method for coating a medical device, comprising:
a. casting upon the medical device a coating formulation comprising:
i. a biodegradable polymeric material;
ii. a bioactive agent; and
iii. a carrier solvent, wherein the bioactive agent and the carrier solvent substantially interact;
b. applying the coating formulation to the medical device; and
c. evaporating the carrier solvent from the coating formulation to form a coating on the medical device, wherein dispersion of the bioactive agent in the coating is heterogeneous.

111. The method of claim 110, wherein the bioactive agent and the carrier solvent substantially interact by an interaction selected from the group consisting of hydrogen bonding, dipole-dipole interactions, ionic-dipole interactions, ionic-ionic interactions and combinations thereof.

112. The method of claim 110, wherein the step of applying the coating formulation is selected from the group consisting of dipping, submersion, spraying, painting, and combinations thereof.

113. The method of claim 110, wherein the step of applying the coating formulation to the medical device is submersion.

114. The method of claim 110, wherein the medical device is submersed in the coating formulation and is removed at a controlled rate, wherein the controlled rate is between 0.1 cm/sec to 10 cm/sec.

115. The method of claim 110, wherein the evaporating step occurs for between about 24 hours to about 48 hours and wherein the temperature of the evaporating step is between about 0° C. to about 50° C.

116. The method of claim 110, wherein the medical device is selected from the group consisting of orthopedic implants, catheters, endotracheal tubes, wound drains, pacemakers, portacaths, and stents or any other medical device manufactured from metal, glasses, tissue, elastomers, plastics and combinations thereof.

117. The method of claim 110, wherein a thickness of the coating on the medical device is between about 0.1 microns to about 500 microns.

118. The method of claim 110, wherein the medical device is a percutaneous medical device.

119. The method of claim 110, wherein the medical device is an intravenous device.

120. The method of claim 110, wherein the medical device is a drainage device.

121. The method of claim 110, wherein the medical device is an endotracheal tube.

122. The method of claim 110, wherein the medical device comprises a material selected from the group consisting of metal, glasses, tissue, elastomers, plastics and combinations thereof.

123. The method of claim 110, wherein the coating eludes the bioactive agent at an elution rate of between about 0.01 μg/cm²/day to about 1000 μg/cm²/day when the medical device is implanted in a patient.

124. The method of claim 110, wherein the biodegradable polymeric material of the coating formulation is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

125. The method of claim 110, wherein the hydrogen bonding solvent of the coating formulation is selected from the group consisting of ethanol, methanol, water and combinations thereof.

126. The method of claim 110, wherein the bioactive agent of the coating formulation is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

127. The method of claim 110, wherein the bioactive agent of the coating formulation is substantially rapidly released immediately upon implantation.

128. The method of claim 110, wherein the carrier solvent of the coating formulation is a solvent mixture in a ratio of 9:1 of chloroform to methanol.

129. A method for preparing a heterogeneous coating, the method comprising:
preparing a mixture by adding a polymeric material and a bioactive agent to a carrier solvent, wherein the carrier solvent comprises a solvent selected from the group consisting of ethanol, methanol, water and combinations thereof;
agitating the mixture between 0° C. and about 75° C. until any of the biodegradable polymeric material and the bioactive agent have dissolved; and
cooling the agitated mixture to form the heterogeneous coating.

130. The method of claim 129, wherein the agitation step is performed by mechanical stirring, magnetic stirring, ultrasonication, shaking, homogenizing, vortexing, or combinations thereof.

131. The method of claim 129, wherein the cooling step occurs at a temperature between 0° C. and 50° C.

132. The method of claim 129, wherein the biodegradable polymeric material is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

133. The method of claim 129, wherein the carrier solvent is selected from the group consisting of ethanol, methanol, water and combinations thereof.

134. The method of claim 129, wherein the bioactive agent is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

135. A coating, comprising:
c) a biodegradable polymeric material;
d) a bioactive agent, wherein dispersion of the bioactive agent in the coating is heterogeneous.

136. The coating of claim 135, wherein the biodegradable polymeric material is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

137. The coating of claim 135, wherein the biodegradable polymeric material is poly(lactide-co-glycolide).

138. The coating of claim 137, wherein an inherent viscosity of the poly(lactide-co-glycolide) is between about 0.1 dL/g to about 1.0 dL/g.

139. The coating of claim 137, wherein an inherent viscosity of the poly(lactide-co-glycolide) is between about 0.4 dL/g to about 0.8 dL/g.

140. The coating of claim 137, wherein the poly(lactide-co-glycolide) comprises between about 10% to about 90% of lactide and about 10% to about 90% of glycolide.

141. The coating of claim 140, wherein the lactide is selected from the group consisting of D-lactide, L-lactide, D,L-lactide and combinations thereof.

142. The coating of claim 137, wherein the poly(lactide-co-glycolide) comprises poly(L-lactide-co-glycolide).

143. The coating of claim 135, wherein an amount of the biodegradable polymeric material in the coating is between about 1% and about 30% by weight to volume of the coating.

144. The coating of claim 135, wherein the bioactive agent is a biguanide.

145. The coating of claim 144, wherein the biguanide is selected from the group consisting of chlorhexidine, polyhexamethylene guanide hydrochloride and combinations thereof.

146. The coating of claim 135, wherein the bioactive agent is a combination of a biguanide and an antibiotic.

147. The coating of claim 135, wherein the bioactive agent is an antibiotic.

148. The coating of claim 135, wherein the bioactive agent is a biocompatible mineral.

149. The coating of claim 135, wherein the bioactive agent is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

150. The coating of claim 135, wherein the bioactive agent is chlorhexidine.

151. The coating of claim 135, wherein the bioactive agent is chlorhexidine free base.

152. The coating of claim 135, wherein the bioactive agent is present in the coating in amounts between about 0.01% to about 50% by weight of the biodegradable polymeric material.

153. The coating of claim 135, wherein the bioactive agent is present in the coating in amounts between about 2% to about 35% by weight of the biodegradable polymeric material.

154. The coating of claim 135, wherein the bioactive agent is present in the coating in amounts between about 5% to about 30% by weight of the biodegradable polymeric material.

155. The coating of claim 135, wherein the bioactive agent is present in the coating in amount of about 25% by weight of the biodegradable polymeric material.

156. The coating of claim 135, wherein the bioactive agent is rapidly released during elution.

157. A coated medical device, comprising:
c) a medical device,
d) a coating on the medical device, wherein the coating comprises:
a. a biodegradable polymeric material; and
b. a bioactive agent, wherein dispersion of the bioactive agent in the coating is heterogeneous.

158. The medical device of claim 157, wherein the biodegradable polymeric material of the coating is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

159. The medical device of claim 157, wherein the biodegradable polymeric material of the coating is poly(lactide-co-glycolide).

160. The medical device of claim 157, wherein the bioactive agent of the coating is selected from the group consisting of antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, hormones, growth factors, cells, bioglasses, hydroxyapatites, phosphates, sulfates and combinations thereof.

161. The medical device of claim 157, wherein the bioactive agent of the coating is substantially rapidly released during elution.

162. The method of claim 157, wherein the coating elutes the bioactive agent at an elution rate of between about 0.01 μg/cm²/day to about 1000 μg/cm²/day when the medical device is implanted in a patient.
